(19) United States
(12) Patent Application Publication
(10) Pub. No.: US 2007/0231345 A1
(43) Pub. Date: Oct. 4, 2007

(54) MEDICAMENT CONTAINING NATURAL LEUCOTRIENE INHIBITORS SINGLY OR IN COMBINATION WITH BIOAVAILABLE ORGANIC SELENIUM SUPPLEMENT FOR HYPERPROLIFERATIVE DERMATOLOGICAL CONDITIONS AND THERAPEUTIC METHODS THEREOF

(76) Inventors: Muhammed Majeed, Piscataway, NJ (US); Subbalakshmi Prakash, Piscataway, NJ (US)

Correspondence Address: SABINSA CORPORATION, 70 ETHEL ROAD WEST, UNIT 6, PISCATAWAY, NJ, 08854, US

(21) Appl. No.: 11/754,646

(22) Filed: May 29, 2007

Related U.S. Application Data
(63) Continuation-in-part of application No. 10/710,778, filed on Aug. 2, 2004.

(51) Int. Cl. A61K 36/324; A61K 31/56

(52) U.S. Cl. 424/195.18; 514/169

(57) ABSTRACT

The current invention discloses a medicament and its use in a method for therapeutically managing hyperproliferative diseases of the skin in particular, psoriasis of the skin, scalp and nails. The medicament includes a dual inhibitor of 5-lipoxygenase and human leukocyte elastase singly or in combination with a bioavailable organic selenium nutritional supplement. In particular, the medicament includes (a) an oral formulation of acetyl-11-keto-beta-boswellic acid in combination with a bioavailable organic selenium supplement dispersed in a pharmaceutical carrier; and (b) a topical formulation of acetyl-11-keto-beta-boswellic acid alone dispersed in a pharmaceutical carrier. The said medicament is shown to be effective for the therapeutic management of clinical psoriasis based on its ability to (i) provide relief of clinical symptoms and (ii) the ability to clear psoriatic lesions as evaluated in 3 months, open, prospective, multi-center, phase III Clinical study.

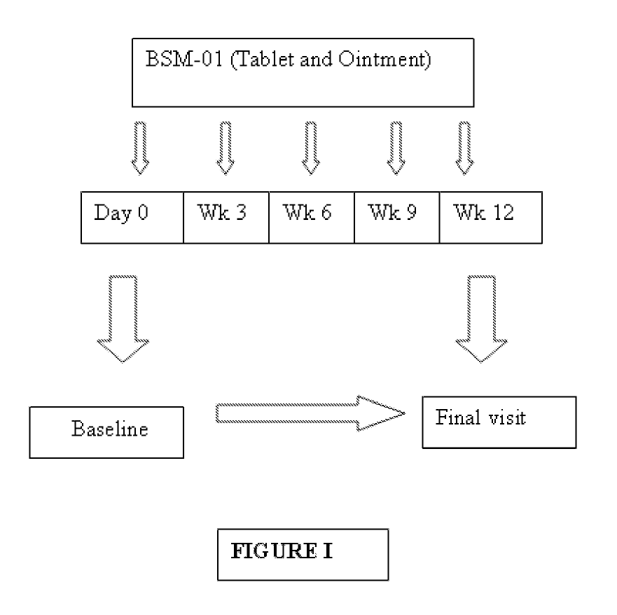

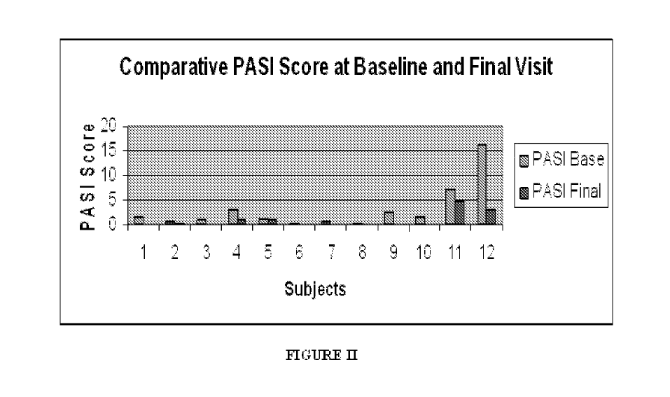

This application is a continuation-in-part of pending U.S. patent application Ser. No. 10/710,778 by the authors, filed on Aug. 02, 2004, for Compositions and methods for the management of hyperproliferative dermatological conditions, the disclosure of which is hereby incorporated by reference.

### BACKGROUND OF THE INVENTION

The present invention in general relates to the management of proliferative dermatological conditions. In particular, the present invention relates to a medicament and its method of use in the prophylactic/therapeutic management of psoriasis of the skin, scalp and nails.

### DESCRIPTION OF THE PRIOR ART

Psoriasis is a non-contagious and chronic skin disease. Psoriasis is a very diverse skin disease that appears in a variety of forms. Each form has distinct characteristics. Typically, people have only one type of psoriasis at a time, but occasionally two or more different types of psoriasis can occur at the same time. Psoriasis can also occasionally change from one form to another.

Psoriasis is generally categorized as (i) Plaque psoriasis (ii) Inverse psoriasis (iii) Erythrodermic psoriasis (iv) Guttate psoriasis (v) Pustular Psoriasis.

Psoriasis is estimated to affect 2-3 percent of the world's population. The National Institutes of Health (NIH) statistics reports that 5.8-7.5 million Americans suffer from psoriasis. Between 10 percent and 30 percent of people with psoriasis also develop psoriatic arthritis. [Gelfand J M, Gladman D D, Mease P J, Smith N, Margolis D J, Nijsten T, Stern R S, Feldman S R, Rolstad T. Epidemiology of psoriatic arthritis in the population of the United States. J Am Acad Dermatol. 2005 October;53(4):573]. With over 3 billion dollars spent annually on the management of psoriasis as reported by Sander H M, Morris L F, Phillips C M, Harrison P E, Menter A. The annual cost of psoriasis. J Am Acad Dermatol. 1993; 28; 422-9, the management of psoriasis has drawn considerable attention in recent years.

Inflammatory cytokines have been implicated in the pathogenesis and pathophysiology of psoriasis. Leukotriene B4 was found to increase approximately 6.6 fold in psoriatic skin lesions [Reilly D M. et al. (2000) Inflammatory mediators in normal, sensitive and diseased skin types. Acta Derm Venereol 80 (3): 171-174]. Iversen L, et al. (1997) in Skin Pharmacol. 10 (4): 169-177 discuss the Significance of leucotriene A4-hydrolase in the pathogenesis of psoriasis. The publication titled “Lesional Elastase activity in psoriasis, contact dermatitis, and atopic dermatitis”by Wiedow O, et al. (1992) in Invest Dermatol. 99 (3): 306-309 reports a high level of elastase activity in the skin lesions of psoriatic cases.

Boswellic acids acids are reported to inhibit 5-lipoxygenase [Safayhi, H. et al. (1992) Boswellic acids:novel, specific, non-redox inhibitors of 5-lipoxygenase. J. Pharmacol.Exp. Ther. 261:1143-1146] and human leukocyte elastase [Safayhi, H. et al. (1997) Inhibition by Boswellic acids of human leukocyte elastase. J. Pharmacol.Exp. Ther. 281:460-463].

Further, low levels of selenium in the blood have been observed in patients with moderate or severe psoriasis.[Corrocher,R et al. (1989) Clin Chim Acta 179 (2): 121-131].A low selenium intake has also been related to the progress of psoriasis by Serwin A B, et al. (1999) Selenium nutritional status and the course of psoriasis. Pol Merkuriusz Lek. 6(35):263-265. The promising effects of selenium-yeast supplementation in the immuno-modulation of psoriatic lesions by increasing the number of CD4+T cells was demonstrated by Harwima, R. J. (1993) Screening of effects of seleno-methionine enriched yeast supplementation on various immunological and chemical parameters of the skin and blood in psoriatic patients. Acta Derm. Venereol. 73(2):88-91.

The inventors of the present invention have thus sought to make use of the immuno-modulatory effects of boswellic acids and bioavailable organic selenium nutritional supplementation to formulate an effective medication for management of clinical psoriasis.

It is thus the principle object of the present invention to develop a medication comprising natural dual inhibitor of 5-lipoxygenase and human leukocyte elastase singly or combined with a bioavailable organic selenium supplementation for the management of hyperproliferative dermatological conditions, in particular psoriasis of the skin, scalp and nails.

It is another object of the present invention to develop a medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement; and (2) a topical formulation comprising acetyl-11-keto-beta-boswellic acid from Boswellia serrata gum resin, congeners or derivatives of acetyl-11-keto-beta-boswellic acid either as single entities or combinations thereof, for the management of hyperproliferative dermatological conditions, in particular psoriasis of the skin, scalp and nails.

It is another object of the present invention to devise an optimal therapeutic method to use the medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement; and (2) a topical formulation comprising acetyl-11-keto-beta-boswellic acid from Boswellia serrata gum resin, congeners or derivatives of acetyl-11-keto-beta-boswellic acid either as single entities or combinations thereof, for the management of hyperproliferative dermatological conditions, in particular psoriasis of the skin, scalp and nails wherein the medication exhibits excellent safety profile as both oral and topical formulations.

Further, the present invention also seeks to develop a medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement and (2) a topical formulation acetyl-11-keto-beta-boswellic acid from Boswellia serrata gum resin, congeners or derivatives of acetyl-11-keto-beta-boswellic acid either as single entities or combinations thereof, for the management of various types of psoriasis including (i) Plaque psoriasis (ii) Inverse psoriasis (iii) Erythrodermic psoriasis (iv) Guttate psoriasis and (v) Pustular Psoriasis.

Still further, the present invention also seeks to evaluate a medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement and (2) a topical formulation comprising acetyl-11-keto-beta-boswellic acid from Boswellia serrata gum resin, congeners or derivatives of acetyl-11-keto-beta-boswellic acid either as single entities or combinations thereof, for the management of hyperproliferative dermatological conditions, in particular psoriasis of the skin, scalp and nails in phased clinical trials.

The present invention fulfills these objectives and provides further related advantages.

### SUMMARY OF THE INVENTION

The present invention discloses a medicament containing natural leucotriene inhibitors singly or in combination with bioavailable organic selenium nutritional supplement for hyperproliferative dermatological conditions and therapeutic methods thereof. The use of the said medication in the management of clinical psoriasis has been proved by phased clinical trials.

The present invention provides the following advantages:
a) A medication comprising a natural dual inhibitor of 5-lipoxygenase and human leukocyte elastase, singly or combined with a bioavailable organic selenium supplementation for the management of hyperproliferative dermatological conditions, in particular psoriasis of the skin, scalp and nails.
b) A medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement; and (2) a topical formulation comprising acetyl-11-keto-beta-boswellic acid from Boswellia serrata gum resin, congeners or derivatives of acetyl-11-keto-beta-boswellic acid either as single entities or combinations thereof, for the management of hyperproliferative dermatological conditions, in particular psoriasis of the skin, scalp and nails.
c) An optimal therapeutic method to use the medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement and (2) a topical formulation comprising acetyl-11-keto-beta-boswellic acid from Boswellia serrata gum resin, congeners or derivatives of acetyl-11-keto-beta-boswellic acid either as single entities or combinations thereof, for the management of hyperproliferative dermatological conditions, in particular psoriasis of the skin, scalp and nails, wherein the medication exhibits an enhanced safety profile of both the topical and oral formulations.
d) A medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement and (2) a topical formulation comprising acetyl-11-keto-beta-boswellic acid from Boswellia serrata gum resin, congeners or derivatives of acetyl-11-keto-beta-boswellic acid either as single entities or combinations thereof, for the management of various types of psoriasis including (i) Plaque psoriasis (ii) Inverse psoriasis (iii) Erythrodermic psoriasis (iv) Guttate psoriasis and (v) Pustular Psoriasis.
e) A medication comprising (1) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement and (2) a topical formulation comprising acetyl-11-keto-beta-boswellic acid from Boswellia serrata gum resin, congeners or derivatives of acetyl-11-keto-beta-boswellic acid either as single entities or combinations thereof, that provides excellent therapeutic results in phased clinical trials for the management of hyperproliferative dermatological conditions, in particular psoriasis of the skin, scalp and nails.

Other features and advantages of the present invention will become apparent from the following more detailed description, taken in conjunction with the accompanying drawings, which illustrate, by way of example, the principle of the invention.

### BRIEF DESCRIPTION OF THE DRAWINGS

Fig. I shows an overview of the study design to evaluate the efficacy and safety of the medicament BSM-01 medicament, including an oral formulation and topical formulation for the management of clinical psoriasis.

Fig. II shows the bar diagram of the PSAI score of the 12 subjects involved in the clinical trial to study the efficacy and safety of the medicament BSM-01 at the baseline and at the end of 12 weeks.

### DETAILED DESCRIPTION OF THE MOST PREFERRED EMBODIMENT

In the most preferred embodiment, the present invention discloses a medicament containing natural leucotriene inhibitors singly or in combination with bioavailable organic selenium nutritional supplement for hyperproliferative dermatological conditions and therapeutic methods thereof.

In one most preferred embodiment, a medicament for the therapeutic management of hyperproliferative dermatological conditions, in particular psoriasis of the skin, scalp and nails, wherein the said medicament comprises a dual inhibitor of 5-lipoxygenase and human leukocyte elastase, namely acetyl-11-keto-beta-boswellic acid from Boswellia serrata gum resin, congeners or derivatives of acetyl-11-keto-beta-boswellic acid either as single entities or combinations thereof, optionally in combination with a bioavailable organic selenium supplement is disclosed. More preferably, the said medicament comprises:
a) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement, dispersed in a pharmaceutically acceptable carrier; and
b) a topical formulation comprising acetyl-11-keto-beta-boswellic acid from Boswellia serrata gum resin, congeners or derivatives of acetyl-11-keto-beta-boswellic acid either as single entities or combinations thereof, dispersed in a pharmaceutically acceptable carrier.

In a preferred embodiment, the said oral formulation contains from about 50 mg to 500 mg of acetyl-11-keto-beta-boswellic acid from Boswellia serrata gum resin, congeners or derivatives of acetyl-11-keto-beta-boswellic acid either as single entities or combinations thereof. In an alternative embodiment, the said oral formulation contains from about 50 mg to 400 mg of acetyl-11-keto-beta-boswellic acid from Boswellia serrata gum resin, congeners or derivatives of acetyl-11-keto-beta-boswellic acid either as single entities or combinations thereof.

In another preferred embodiment, the oral formulation of the said medicament comprises 50 micrograms to 200 micrograms of bioavailable organic selenium nutritional supplement selected from the group consisting of selenomethionine, methylselenocysteine, derivatives of selenomethionine, derivatives of methylselenocysteine, isomeric peptides of selenomethionine, isomeric peptides of methylselenocysteine, high selenium yeast and selenium enriched vegetables. In an alternative embodiment, the said oral formulation comprises 10 micrograms to 100 micrograms of bioavailable organic selenium nutritional supplement derived from L (+) Selenomethionine.

In yet another preferred embodiment the topical formulation of the said medicament contains from about 50 mg to 400 mg of acetyl-11-keto-beta-boswellic acid from Boswellia serrata gum resin, congeners or derivatives of acetyl-11-keto-beta-boswellic acid either as single entities or combinations thereof.

In yet another preferred embodiment, the total daily oral dose of acetyl-11-keto-beta-boswellic acid from Boswellia serrata gum resin, congeners or derivatives of acetyl-11-keto-beta-boswellic acid either as single entities or combinations thereof, is from about 50 mg to about 2000 mg and, the said oral dose is divided into a plurality of doses.

In yet another preferred embodiment, the total daily oral dose of bioavailable organic selenium supplement is from about 10 micrograms to about 400 micrograms, and the said oral dose is divided into a plurality of doses.

In yet another preferred embodiment, the total daily topical dose of acetyl-11-keto-beta-boswellic acid from Boswellia serrata gum resin, congeners or derivatives of acetyl-11-keto-beta-boswellic acid either as single entities or combinations thereof, is about 10 mg to about 1600 mg and, the said topical dose is divided into a plurality of doses.

In one other most preferred embodiment, a method of therapeutically managing hyperproliferative dermatological conditions, in particular psoriasis of the skin, scalp and nails, said method comprising the administration of a medicament comprising a dual inhibitor of 5-lipoxygenase and human leukocyte elastase namely, acetyl-11-keto-beta-boswellic acid from Boswellia serrata gum resin, congeners or derivatives of acetyl-11-keto-beta-boswellic acid either as single entities or combinations thereof, optionally in combination with a bioavailable organic selenium supplement is described.

In a preferred embodiment, the method of therapeutically managing hyperproliferative dermatological conditions in particular psoriasis of the skin, scalp and nails, said method involving the administration of a medicament comprising:
a) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement, dispersed in a pharmaceutically acceptable carrier; and
b) a topical formulation comprising acetyl-11-keto-beta-boswellic acid from Boswellia serrata gum resin, congeners or derivatives of acetyl-11-keto-beta-boswellic acid either as single entities or combinations thereof, dispersed in a pharmaceutically acceptable carrier.

In a preferred embodiment, the method of therapeutically managing hyperproliferative dermatological conditions, in particular psoriasis of the skin, scalp and nails involves administering the said oral formulation containing from about 50 mg to 500 mg of acetyl-11-keto-beta-boswellic acid from Boswellia serrata gum resin, congeners or derivatives of acetyl-11-keto-beta-boswellic acid either as single entities or combinations thereof. In an alternative embodiment, the said oral formulation contains from about 50 mg to 400 mg of acetyl-11-keto-beta-boswellic acid from Boswellia serrata gum resin, congeners or derivatives of acetyl-11-keto-beta-boswellic acid either as single entities or combinations thereof.

In another preferred embodiment, the method of therapeutically managing hyperproliferative dermatological conditions, in particular psoriasis of the skin, scalp and nails involves administering the said oral formulation containing from about 50 micrograms to 200 micrograms of bioavailable organic selenium nutritional supplement selected from the group consisting of selenomethionine, methylselenocysteine, derivatives of selenomethionine, derivatives of methylselenocysteine, isomeric peptides of selenomethionine, isomeric peptides of methylselenocysteine, high selenium yeast and selenium enriched vegetables. In an alternative embodiment, the method of therapeutically managing hyperproliferative dermatological conditions, in particular psoriasis of the skin, scalp and nails involves administering the said oral formulation containing about 10 micrograms to 100 micrograms of bioavailable organic selenium nutritional supplement derived from L (+) Selenomethionine.

In another preferred embodiment, the method of therapeutically managing hyperproliferative dermatological conditions, in particular psoriasis of the skin, scalp and nails involves administering the said topical formulation containing from about 50mg to 400 mg of acetyl-11-keto-beta-boswellic acid from Boswellia serrata gum resin, congeners or derivatives of acetyl-11-keto-beta-boswellic acid either as single entities or combinations thereof.

In yet another preferred embodiment, the method of therapeutically managing hyperproliferative dermatological conditions, in particular psoriasis of the skin, scalp and nails involves total daily oral dose of acetyl-11-keto-beta-boswellic acid from Boswellia serrata gum resin, congeners or derivatives of acetyl-11-keto-beta-boswellic acid either as single entities or combinations thereof, from about 50 mg to about 2000 mg and, the said oral dose is divided into a plurality of doses.

In yet another preferred embodiment, the method of therapeutically managing hyperproliferative dermatological conditions, in particular psoriasis of the skin, scalp and nails involves a total daily oral dose of bioavailable selenium organic supplement from about 10 micrograms to about 400 micrograms and, the said oral dose is divided into a plurality of doses.

In yet another preferred embodiment, the method of therapeutically managing hyperproliferative dermatological conditions, in particular psoriasis of the skin, scalp and nails involves a total daily topical dose of acetyl-11-keto-beta-boswellic acid from Boswellia serrata gum resin, congeners or derivatives of acetyl-11-keto-beta-boswellic acid either as single entities or combinations thereof, is from about 10 mg to about 1600 mg and, the said topical dose is divided into a plurality of doses.

The most preferred embodiment of the present invention namely, a medicament (BSM-01) containing natural leukotriene inhibitors optionally in combination with bioavailable organic selenium for hyperproliferative dermatological conditions and therapeutic methods thereof as discussed herein above are further illustrated through specific examples included herein below. BSM-01 medicament includes (a) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement, dispersed in a pharmaceutically acceptable carrier; and (b) a topical formulation comprising acetyl-11-keto-beta-boswellic acid from Boswellia serrata gum resin, congeners or derivatives of acetyl-11-keto-beta-boswellic acid either as single entities or combinations thereof, dispersed in a pharmaceutically acceptable carrier. The oral formulation is in the form of a tablet and the topical formulation is in the form of an ointment.

### EXAMPLE I

OBJECTIVE: To study the efficacy and safety of the medicament BSM-01, including an oral formulation and topical formulation for the management of clinical psoriasis through (a) Relief of symptoms and (b) Extent of clearance of psoriatic lesions.

STUDY DESIGN: A 3 months open, prospective, multi-center, phase III clinical study. Male and female subjects were administered BSM-01 medicament comprising an oral formulation in the form of tablets and a topical application in the form of ointment three times a day for 12 weeks. A total number of 12 subjects were involved in the study. An overview of the study design is illustrated in Fig. I.

Outline of Study Procedures

TABLE I

| | Screening/ | Visit 1- | Visit 2- | Visit 3- | Visit 4- |
|---|---|---|---|---|---|
| | Baseline | Week 3 | Week 6 | Week 9 | Week 12 |
| Procedure | | | | | |
| Informed | X | | | | |
| Consent | | | | | |
| Inclusion/ | X | | | | |
| Exclusion | | | | | |
| Demographics | X | | | | |
| Physical | X | X | X | X | X |
| Examination | | | | | |
| Concomitant | X | X | X | X | X |
| Therapy | | | | | |
| Clinical | X | | X | | X |
| Photographs | | | | | |
| Skin Biopsy | X | | | | X |
| Clinical | X | X | X | X | X |
| Examination | | | | | |
| Laboratory | | | | | |
| Tests | | | | | |
| Liver function | X | | | | X |
| tests | | | | | |
| Renal function | X | | | | X |
| tests | | | | | |
| Selenium level | X | | | | X |
| Adverse Events | | X | X | X | X |

Inclusion Criteria
1. Patients aged 18-65 years
2. Patients with Mild to Moderate Psoriasis
3. Patients who have not used corticosteroids or (if used with a one month washout period)
4. Patients who have signed the informed consent form.

Exclusion Criteria
1. Subjects who have more than 50 percent of body surface area covered by psoriasis lesions
2. Subjects on anticoagulant therapy
3. Subjects who have used immunosuppressive medication within past 2 months
4. Subjects with other skin lesions (actinic ketoses or lentigo) or photo damaged skin
5. Subjects who have used systemic or intralesional therapy or photo (chemo) therapy for psoriasis in the previous two months
6. Subjects with concomitant bacterial, fungal, or vital skin infections
7. Subjects who are pregnant or sexually active women who do not use contraception
8. Subjects who show non-adherence to the treatment
9. Subjects who have a history of renal disease, hepatobiliary disease, malignancy, hypertension or recurrent acute infection
10. History of any other evidence of severe illness or any other conditions that would make the subject, in the opinion of the investigator, unsuitable for the study

Scoring System

The PASI (Psoriasis Activity and Severity Index) scoring system was used to assess patients with psoriasis over time and to monitor their response to therapy.

Elements
(1) Body regions as percent of body surface area (Table II)
(2) Extent of body region affected (Table III)

(3) Extent of psoriatic changes (Table IV)

TABLE II

| Body Region | Code | Percent Body Surface Area |
|---|---|---|
| Head | H | 10% |
| Trunk | T | 20% |
| Upper extremities | U | 30% |
| Lower extremities | L | 40% |

TABLE III

| Portion of Body Region Affected | Extent Indicator |
|---|---|
| 0-5% | 0 |
| 5-25% | 1 |
| 25-45% | 2 |
| 45-55% | 3 |
| 55-75% | 4 |
| 75-95% | 5 |
| 95-100% | 6 |

TABLE IV

| Changes in Psoriasis | Code | Extent |
|---|---|---|
| Erythema | E | 0-4 |
| Infiltration | I | 0-4 |
| Desquamation | D | 0-4 |

PASI=
=SUM ((percent BSA in body region)*((extent erythema in region)+(extent infiltration in region)+(extent desquamation in region))*(extent of body region affected))=
=((0.1*((erythema head)+(infiltration head)+(desquamation head))*(extent of head affected))+((0.2*((erythema trunk)+(infiltration trunk)+(desquamation trunk))*(extent of trunk affected))+((0.3*((erythema upper extremities)+(infiltration upper extremities)+(desquamation upper extremities))*(extent of upper extremities affected))+((0.4*((erythema lower extremities)+(infiltration lower extremities)+(desquamation lower extremities))*(extent of lower extremities affected))

Interpretation:

Minimum score=0

Maximum score=72

Ethics and Regulatory Considerations
1. The study was conducted according to “Good Clinical Practice and the declaration of Helsinki”, covering the Local rules and regulations of the country.
2. The study was conducted only by getting the clearance from the ethical committee.
3. Informed consent was obtained from all patients as per the guidelines under the declaration of Helsinki

Study Product and Administration

Dosage and Administration

BSM-01 [tablets (Oral application)+ointment (topical application)] was administered concurrently thrice a day for 12 weeks

Concomitant Medication/Treatment

At each study visit/contact the investigator questioned the subject about any medication taken. Any immuno-suppressants or other immune modifying drugs or treatment other than the study drug and any antipyretics administered at any time during the period starting 2 weeks prior to the first dose of the study drug and ending 2 weeks after the last dose of the study drug was recorded in the CRF with trade name and/or generic name of the medication.

Adverse Events

All adverse reactions reported and seen at each visit were carefully documented.

Results and Discussion

Table V demonstrates the PASI score of 12 subjects at the baseline and the end of 12 weeks. Figure II is the bar diagram of the PSAI score of the 12 subjects at the baseline and at the end of 12 weeks.

TABLE V

| Subject Code | PASI Base | PASI Final |
|---|---|---|
| 1 | 1.6 | 0 |
| 2 | 0.6 | 0.4 |
| 3 | 0.8 | 0 |
| 4 | 3 | 0.8 |
| 5 | 1.2 | 0.8 |
| 6 | 0.4 | 0 |
| 7 | 0.7 | 0 |
| 8 | 0.3 | 0 |
| 9 | 2.4 | 0 |
| 10 | 1.6 | 0 |
| 11 | 7.2 | 4.8 |
| 12 | 16.2 | 3.2 |

It was clear that in all 12 subjects, there was a significant decrease in PASI Score. Out of the 12 volunteers, seven (58.3%) had a drop in PASI Score to zero at the end of the 12^{th} week.

Table VI shows the comparative haematology of the 12 subjects at baseline and final visit.

TABLE VI

| | | |
|---|---|---|
| Subject | ESR (mm in I hour) | Hemoglobin (g/dl) |

| Code | 0 Week | 12 Week | 0 Week | 12 Week |
|---|---|---|---|---|
| 1 | 20 | 26 | 14.5 | 13.4 |
| 2 | 7 | 32 | 13.4 | 13.4 |
| 3 | 5 | 12 | 16.2 | 15.4 |
| 4 | 5 | 27 | 13.3 | 12.8 |
| 5 | 24 | 22 | 14.1 | 14.8 |
| 6 | 8 | 6 | 12.6 | 13.5 |
| 7 | 17 | 38 | 12.5 | 13.4 |
| 8 | 5 | 3 | 14.7 | 15.2 |
| 9 | 12 | 7 | 13.2 | 12.5 |
| 10 | 25 | 15 | 13.6 | 13.7 |
| 11 | 20 | 28 | 12.9 | 13.5 |
| 12 | 12 | 20 | 14.2 | 13.8 |

Tables VIIA, VIIB and VIIC shows the Comparative Liver Functional Tests at Baseline and Final Visit

TABLE VII A

| | Serum Total | | Serum Direct | | Serum Indirect | |
|---|---|---|---|---|---|---|
| | Bilirubin | | Bilirubin | | Bilirubin | |
| Subject | (mg/dl) | | (mg/dl) | | (mg/dl) | |
| Code | 0 Week | 12 Week | 0 Week | 12 Week | 0 Week | 12 Week |
| 1 | 1.5 | 1.4 | 0.4 | 0.2 | 1.1 | 1.2 |
| 2 | 1.1 | 0.4 | 0.3 | 0.1 | 0.8 | 0.3 |
| 3 | 0.8 | 0.7 | 0.3 | 0.2 | 0.5 | 0.5 |
| 4 | 0.8 | 0.9 | 0.3 | 0.2 | 0.5 | 0.7 |
| 5 | 0.6 | 0.6 | 0.2 | 0.2 | 0.4 | 0.4 |
| 6 | 0.7 | 0.9 | 0.3 | 0.2 | 0.4 | 0.7 |
| 7 | 0.8 | 0.9 | 0.6 | 0.6 | 0.2 | 0.3 |
| 8 | 0.9 | 0.8 | 0.6 | 0.6 | 0.3 | 0.2 |
| 9 | 0.8 | 0.8 | 0.4 | 0.5 | 0.4 | 0.3 |
| 10 | 0.8 | 0.9 | 0.4 | 0.5 | 0.4 | 0.4 |
| 11 | 0.6 | 0.8 | 0.4 | 0.4 | 0.2 | 0.4 |
| 12 | 0.9 | 0.9 | 0.6 | 0.6 | 0.3 | 0.3 |

TABLE VII B

| | Serum Protein | | | | | | | |
|---|---|---|---|---|---|---|---|---|
| | Total | | Serum Albumin | | Serum Globulin | | | |
| Subject | (gm/dl) | | (gm/dl) | | (gm/dl) | | A/G Ratio | |
| Code | 0 Week | 12 Week | 0 Week | 12 Week | 0 Week | 12 Week | 0 Week | 12 Week |
| 1 | 7.7 | 7 | 3.5 | 3.5 | 3.4 | 3.5 | 1.26 | 1 |
| 2 | 8.4 | 7.6 | 5 | 4.1 | 3.4 | 3.5 | 1.47 | 1.17 |
| 3 | 7.9 | 7.2 | 4.6 | 4.4 | 3.3 | 2.8 | 1.39 | 1.57 |
| 4 | 7.5 | 7.4 | 4.5 | 4.3 | 3 | 3.1 | 1.5 | 1.39 |
| 5 | 7.9 | 7.5 | 4.9 | 4.3 | 3 | 3.2 | 1.63 | 1.34 |
| 6 | 7.4 | 7.2 | 4.4 | 4.9 | 3 | 2.3 | 1.47 | 2.13 |
| 7 | 6.3 | 6.6 | 4 | 4.2 | 2.3 | 2.4 | 1.74 | 1.75 |
| 8 | 6.8 | 7 | 4.2 | 3.7 | 2.6 | 3.3 | 1.62 | 1.12 |
| 9 | 6.7 | 6.8 | 4.4 | 4.6 | 2.3 | 2.2 | 1.91 | 1.64 |
| 10 | 7.5 | 6.6 | 4 | 2.8 | 3.5 | 3.8 | 1.14 | 0.74 |
| 11 | 7.2 | 6.6 | 4 | 3 | 3.2 | 3.6 | 1.25 | 0.83 |
| 12 | 7.3 | 7 | 4.3 | 4 | 3 | 3 | 1.43 | 1.33 |

TABLE VII C

| | | | SGPT | | Alkaline | | GGT | |
|---|---|---|---|---|---|---|---|---|
| Subject | SGOT (U/L) | | (U/L) | | Phosphatase (U/L) | | (U/L) | |
| Code | 0 Week | 12 Week | 0 Week | 12 Week | 0 Week | 12 Week | 0 Week | 12 Week |
| 1 | 18 | 21 | 23 | 13 | 163 | 175 | 12 | 14 |
| 2 | 30 | 33 | 25 | 35 | 190 | 132 | 10 | 12 |
| 3 | 23 | 25 | 32 | 29 | 238 | 245 | 37 | 31 |
| 4 | 22 | 19 | 40 | 25 | 220 | 225 | 11 | 5 |
| 5 | 32 | 30 | 21 | 15 | 185 | 191 | 25 | 20 |
| 6 | 30 | 29 | 28 | 25 | 312 | 379 | 13 | 12 |
| 7 | 26 | 25 | 32 | 35 | 74 | 110 | — | — |
| 8 | 29 | 16 | 33 | 20 | 76 | 72 | — | — |
| 9 | 30 | 26 | 28 | 25 | 80 | 100 | — | — |
| 10 | 22 | 27 | 29 | 26 | 109 | 134 | — | — |
| 11 | 31 | 25 | 31 | 24 | 103 | 151 | — | — |
| 12 | 27 | 30 | 25 | 27 | 70 | 76 | — | — |

Tables VIII A and VIII B show the comparative Renal Functional Tests of the twelve subjects at the Baseline and Final Visit.

TABLE VIII A

| | BUN | | Serum Creatinine | | Uric Acid | |
|---|---|---|---|---|---|---|
| Subject | (mg/dl) | | (mg/dl) | | (mg/dl) | |
| Code | 0 Week | 12 Week | 0 Week | 12 Week | 0 Week | 12 Week |
| 1 | 10.7 | 10.2 | 0.51 | 0.6 | 4.2 | 3.5 |
| 2 | 9.8 | 8.8 | 0.6 | 0.8 | 5 | 3.9 |
| 3 | 11.6 | 11.7 | 0.72 | 0.8 | 3 | 5.2 |
| 4 | 15.4 | 12.1 | 0.97 | 0.8 | 4.7 | 4.7 |
| 5 | 13.5 | 10.7 | 0.9 | 0.8 | 5 | 4.3 |
| 6 | 9.1 | 10.7 | 0.8 | 0.7 | 5.6 | 5.8 |
| 7 | 10.5 | 10.6 | 1 | 1.1 | 5.7 | 5.2 |
| 8 | 11.1 | 10.4 | 1 | 1.1 | 7 | 7 |
| 9 | 10.3 | 10.8 | 1.4 | 1.1 | 6.6 | 6.3 |
| 10 | 15.1 | 10.1 | 1.1 | 1.2 | 5.8 | 5.8 |
| 11 | 10 | 12.7 | 1.1 | 1.2 | 3.4 | 4.3 |
| 12 | 12.5 | 13.9 | 1.1 | 1.1 | 4.8 | 5.2 |

TABLE VIII B

| | | | Potassium | | | | | | Inorganic | |
|---|---|---|---|---|---|---|---|---|---|---|
| Subject | Sodium (mEq/l) | | (mEq/l) | | Chloride (mEq/l) | | Calcium (mg/dl) | | Phosphorus (mg/dl) | |
| Code | 0 Week | 12 Week | 0 Week | 12 Week | 0 Week | 12 Week | 0 Week | 12 Week | 0 Week | 12 Week |
| 1 | 143 | 139 | 4.8 | 4.2 | 101 | 96 | 10.6 | 8.5 | 3.8 | 3.4 |
| 2 | 146 | 143 | 4.2 | 4 | 99 | 103 | 8.7 | 10.6 | 2.8 | 4.3 |
| 3 | 142 | 139 | 4.3 | 4 | 99 | 105 | 9.8 | 8.9 | 3.9 | 4.9 |
| 4 | 142 | 140 | 4.4 | 3.9 | 97 | 104 | 10 | 10.7 | 3.7 | 3 |
| 5 | 142 | 141 | 4.4 | 4.1 | 98 | 100 | 10.3 | 10.2 | 2.6 | 4.1 |
| 6 | 140 | 143 | 4.1 | 4.1 | 98 | 103 | 10 | 10.7 | 3 | 4.7 |
| 7 | 140 | 140 | 4.3 | 4.1 | 92 | 95 | — | — | — | — |
| 8 | 142 | 142 | 4.8 | 4.4 | 94 | 93 | — | — | — | — |
| 9 | 140 | 143 | 4.1 | 4.3 | 93 | 94 | — | — | — | — |
| 10 | 143 | 141 | 4.2 | 4.6 | 95 | 93 | — | — | — | — |
| 11 | 140 | 141 | 3.6 | 4 | 90 | 94 | — | — | — | — |
| 12 | 141 | 140 | 4.1 | 4 | 94 | 93 | — | — | — | — |

The advantages of the BSM-01 medicament include:
a) BSM-01 medicament comprises both (a) an oral formulation comprising acetyl-11-keto-beta-boswellic acid from Boswellia serrata gum resin, congeners or derivatives of acetyl-11-keto-beta-boswellic acid either as single entities or combinations thereof, further in combination with a bioavailable organic selenium nutritional supplement, dispersed in a pharmaceutically acceptable carrier; and
   (b) a topical formulation comprising acetyl-11-keto-beta-boswellic acid from Boswellia serrata gum resin, congeners or derivatives of acetyl-11-keto-beta-boswellic acid either as single entities or combinations thereof, dispersed in a pharmaceutically acceptable carrier. The oral and topical formulations may be administered concurrently to provide an enhanced therapeutic management of hyperproliferative dermatological conditions, in particular psoriasis of the skin, scalp and nails.
   b) BSM-01 medicament exhibits an enhanced safety profile and the oral and topical formulations of the said medicament do not create untoward side effects upon administration.
   c) BSM-01 medicament does not cause significant changes in haematology, liver functional tests and renal functional tests.
   d) BSM-01 medicament bears with it an innate advantage of dual immuno-modulatory effects of acetyl-11-keto-beta boswellic acid and bio available organic selenium supplementation.
   e) BSM-01 medicament exhibits therapeutic potential towards all forms of psoriasis including including (i) Plaque psoriasis (ii) Inverse psoriasis (iii) Erythrodermic psoriasis (iv) Guttate psoriasis and (v) Pustular Psoriasis.

While the invention has been described with reference to a preferred embodiment, it is to be clearly understood by those skilled in the art that the invention is not limited thereto. Rather, the scope of the invention is to be interpreted only in conjunction with the appended claims.

We claim

1. A medicament for the therapeutic management of hyperproliferative dermatological conditions, in particular psoriasis of the skin, scalp and nails, wherein the said medicament comprises a dual inhibitor of 5-lipoxygenase and human leukocyte elastase derived from Boswellia serrata gum resin, either singly or in combination with a bioavailable organic selenium supplement.

2. A medicament for the therapeutic management of hyperproliferative dermatological conditions, in particular psoriasis of the skin, scalp and nails, wherein the said medicament comprises:
c) an oral formulation comprising acetyl-11-keto-beta-boswellic acid derived from Boswellia serrata gum resin, congeners or derivatives of acetyl-11-keto-beta-boswellic acid either as single entities or combinations thereof, further in combination with a bioavailable organic selenium nutritional supplement, dispersed in a pharmaceutically acceptable carrier; and
d) a topical formulation comprising acetyl-11-keto-beta-boswellic acid derived from Boswellia serrata gum resin, congeners or derivatives of acetyl-11-keto-beta-boswellic acid either as single entities or combinations thereof, dispersed in a pharmaceutically acceptable carrier.

3. The medicament according to claim 2, wherein the said oral formulation contains from about 50 mg to 500 mg of acetyl-11-keto-beta boswellic acid derived from Boswellia serrata gum resin, congeners or derivatives of acetyl-11-keto-beta-boswellic acid either as single entities or combinations thereof.

4. The medicament according to claim 2, wherein the said oral formulation contains from about 50 mg to 400 mg of acetyl-11-keto-beta boswellic acid derived from Boswellia serrata gum resin, congeners or derivatives of acetyl-11-keto-beta-boswellic acid either as single entities or combinations thereof.

5. The medicament as in any one of claims 1 or 2, in which the said medicament comprises 50 micrograms to 200 micrograms of bioavailable organic selenium nutritional supplement selected from the group consisting of selenomethionine, methylselenocysteine, derivatives of selenomethionine, derivatives of methylselenocysteine, isomeric peptides of selenomethionine, isomeric peptides of methylselenocysteine, high selenium yeast and selenium enriched vegetables.

6. The medicament as in any one of claims 1 or 2, in which the said medicament comprises 10 micrograms to 100 micrograms of bioavailable organic selenium nutritional supplement derived from L (+) Selenomethionine.

7. The medicament according to claim 2, wherein the said topical formulation contains from about 50mg to 400 mg of acetyl-11-keto-beta-boswellic acid derived from Boswellia serrata gum resin, congeners or derivatives of acetyl-11-keto-beta-boswellic acid either as single entities or combinations thereof.

8. The medicament according to claim 2, wherein the total daily oral dose of acetyl-11-keto-beta-boswellic acid from Boswellia serrata gum resin, congeners or derivatives of acetyl-11-keto-beta-boswellic acid either as single entities or combinations thereof is about 50 mg to about 2000 mg and the said oral dose is divided into a plurality of doses.

9. The medicament according to claim 2, wherein the total daily oral dose of bioavailable selenium organic supplement is from about 10 micrograms to about 400 micrograms and the said oral dose is divided into a plurality of doses.

10. The medicament according to claim 2, in which the total daily topical dose of acetyl-11-keto-beta-boswellic acid from Boswellia serrata gum resin, congeners or derivatives of acetyl-11-keto-beta-boswellic acid either as single entities or combinations thereof is about 10 mg to about 1600 mg and the said topical dose is divided into a plurality of doses.

11. The medicament as in one of claims 1, 2, 3, 4, 7, 8, 9 or 10, in which the said medicament is effective in (a) providing relief of symptoms associated with clinical psoriasis; and (b) clearing psoriatic lesions of the skin, scalp and nails, as scored by the Psoriasis Activity and Severity Index System in clinical studies.

12. The medicament as in one of claims 1, 2, 3, 4, 7, 8, 9 or 10, in which the said medicament is effective in (a) providing relief of symptoms associated with clinical psoriasis; and (b) clearing psoriatic lesions of the skin, scalp and nails, as scored by the Psoriasis Activity and Severity Index System in clinical studies without affecting haematological, liver function and renal function parameters.

13. The medicament as in one of claims 1, 2, 3, 4, 7, 8, 9 or 10, in which the said medicament is effective with an enhanced safety profile, in (a) providing relief of symptoms associated with clinical psoriasis; and (b) clearing psoriatic lesions of the skin, scalp and nails, as scored by the Psoriasis Activity and Severity Index System in clinical studies.

14. A method of therapeutically managing hyperproliferative dermatological conditions in particular psoriasis of the skin, scalp and nails, said method comprising the administration of a medicament comprising a dual inhibitor of 5-lipoxygenase and human leukocyte elastase derived from Boswellia serrata gum resin either singly or in combination with a bioavailable organic selenium supplement.

15. A method of therapeutically managing hyperproliferative dermatological conditions in particular psoriasis of the skin, scalp and nails, said method comprising the administration of a medicament comprising:
e) an oral formulation comprising acetyl-11-keto-beta-boswellic acid derived from Boswellia serrata gum resin, congeners or derivatives of acetyl-11-keto-beta-boswellic acid either as single entities or combinations thereof, further in combination with a bioavailable organic selenium nutritional supplement, dispersed in a pharmaceutically acceptable carrier; and
f) a topical formulation comprising acetyl-11-keto-beta-boswellic acid derived from Boswellia serrata gum resin, congeners or derivatives of acetyl-11-keto-beta-boswellic acid either as single entities or combinations thereof, dispersed in a pharmaceutically acceptable carrier.

16. The method according to according to claim 15, wherein the said oral formulation contains from about 50 mg to 500 mg of acetyl-11-keto-beta boswellic acid derived from Boswellia serrata gum resin, congeners or derivatives of acetyl-11-keto-beta-boswellic acid either as single entities or combinations thereof.

17. The method according to claim 15, wherein the said oral formulation contains from about 50 mg to 400 mg of acetyl-11-keto-beta boswellic acid derived from Boswellia serrata gum resin, congeners or derivatives of acetyl-11-keto-beta-boswellic acid either as single entities or combinations.

18. The method as in any one of claims 14 or 15, in which the said medicament comprises 50 micrograms to 200 micrograms of bioavailable organic selenium nutritional supplement selected from the group consisting of selenomethionine, methylselenocysteine, derivatives of selenomethionine, derivatives of methylselenocysteine, isomeric peptides of selenomethionine, isomeric peptides of methylselenocysteine, high selenium yeast and selenium enriched vegetables in a pharmaceutical carrier.

19. The method as in any one of claims 14 or 15, in which the said medicament comprises 10 micrograms to 100 micrograms of bioavailable organic selenium nutritional supplement derived from L (+) Selenomethionine in a pharmaceutical carrier.

20. The method according to claim 15, wherein the said topical formulation contains from about 50 mg to 400 mg of acetyl-11-keto-beta-boswellic acid derived from Boswellia serrata gum resin, congeners or derivatives of acetyl-11-keto-beta-boswellic acid either as single entities or combinations thereof.

21. The method according to claim 15, wherein the total daily oral dose of acetyl-11-keto-beta-boswellic acid from Boswellia serrata gum resin, congeners or derivatives of acetyl-11-keto-beta-boswellic acid either as single entities or combinations thereof, is about 50 mg to about 2000 mg and, the said oral dose is divided into a plurality of doses.

22. The method according to claim 15, wherein the total daily oral dose of bioavailable selenium organic supplement is from about 10 micrograms to about 400 micrograms, and the said oral dose is divided into a plurality of doses.

23. The method according to claim 15, wherein the total daily topical dose of acetyl-11-keto-beta-boswellic acid from Boswellia serrata gum resin, congeners or derivatives of acetyl-11-keto-beta-boswellic acid either as single entities or combinations thereof, is about 10 mg to about 1600 mg and, the said topical dose is divided into a plurality of doses.

24. The method as in one of claims 14, 15, 16, 17, 20, 21, 22 or 23, in which the said medicament is effective in (a) providing relief of symptoms associated with clinical psoriasis; and (b) clearing psoriatic lesions of the skin, scalp and nails, as scored by the Psoriasis Activity and Severity Index System in clinical studies.

25. The method as in one of claims 14, 15, 16, 17, 20, 21, 22 or 23, in which the said medicament is effective in (a) providing relief of symptoms associated with clinical psoriasis; and (b) clearing psoriatic lesions of the skin, scalp and nails, as scored by the Psoriasis Activity and Severity Index System in clinical studies without affecting haematological, liver function and renal function parameters.

26. The method as in one of claims 14, 15, 16, 17, 20, 21, 22 or 23, in which the said medicament is effective, with an enhanced safety profile in (a) providing relief of symptoms associated with clinical psoriasis; and (b) clearing psoriatic lesions of the skin, scalp and nails, as scored by the Psoriasis Activity and Severity Index System in clinical studies.
